(19) United States
(12) Patent Application Publication
(10) Pub. No.: US 2009/0287236 A1
(43) Pub. Date: Nov. 19, 2009

(54) ENDOSCOPIC ROTARY ACCESS NEEDLE

(75) Inventors: Gregory J. Bakos, Mason, OH (US); William D. Fox, New Richmond, OH (US); John M. Mugan, Galway (IE); Damien J. Ryan, Galway (IE)

Correspondence Address: K&L GATES LLP, 535 SMITHFIELD STREET, PITTSBURGH, PA, 15222, US

(73) Assignee: Ethicon Endo-Surgery, Inc., Cincinnati, OH (US)

(21) Appl. No.: 12/122,031

(22) Filed: May 16, 2008

(51) Int. Cl. A61B 17/32; A61L 2/00

(52) U.S. Cl. 606/180; 422/40

(57) ABSTRACT

Various methods and devices are provided for cutting an opening through tissue. In one embodiment, a tissue-penetrating device is provided and includes a flexible elongate needle shaft that has a distal end with a circumferentially extending tissue-cutting edge formed thereon. A proximal end of the elongate needle shaft interfaces with a rotary control member for selectively applying a rotary motion to the elongate needle shaft. When the rotating tissue-cutting edge is brought into contact with target tissue, the tissue-cutting edge cuts an opening through the tissue. The device may have a stylet that has a blunt end that can be adjusted to protrude out of the distal end of the elongate needle shaft out beyond the tissue-cutting edge and is biased inward into the elongate needle shaft when the blunt end is brought into contact with the tissue to expose the tissue-cutting edge to the tissue. The stylet may be used as a guide wire after the hole has been cut through the tissue. An outer sheath may be provided to selectively cover the distal end of the elongate needle shaft and blunt end of the stylet. The outer sheath may have a selectively expandable member thereon that may be used to expand the cut hole

### FIELD OF THE INVENTION

The present invention relates, in general, to methods and devices for forming passages through tissue, and more particularly, to rotary endoscopic needle arrangements and methods.

### BACKGROUND OF THE INVENTION

Laparoscopic surgery is one type of minimally invasive surgery in which a surgeon uses numerous trocar ports to access and visualize the tissue site of interest within the abdominal cavity of a fully anesthetized patient. The benefits of laparoscopic surgery, as compared to open incisional, abdominal surgery, include less pain, shorter recovery time, less scarring, and lower cost. Another way to access the abdominal cavity, however, is via natural openings (mouth, anus, vagina, urethra) of the body and through the peritoneal lining of the abdominal cavity. Obviously, the size and shape of instruments that may be passed through a bodily lumen in order to perform a medical procedure in the abdominal cavity are greatly restricted due to the anatomical properties of the lumen.

General surgeons, gastroenterologists, and other medical specialists routinely use flexible endoscopes for intraluminal (within the lumen of the alimentary canal) examination and treatment of the upper gastrointestinal (GI) tract, via the mouth, and the lower GI tract, via the anus. In these procedures, the physician pushes the flexible endoscope into the lumen, periodically pausing to articulate the distal end of the endoscope using external control knobs, to redirect the distal tip of the endoscope. In this way, the physician may navigate the crooked passageway of the upper GI past the pharynx, through the esophagus and gastro esophageal junction, and into the stomach. The physician must take great care not to injure the delicate mucosal lining of the lumen, which generally may stretch open to a diameter in the range of about 15-25 mm, but normally has a non-circular cross sectional configuration when relaxed.

During such translumenal procedures, a through-passage must be formed in the stomach wall or in the gastrointestinal tract to access the peritoneal cavity. One device often used to form such a puncture is a needle knife which is inserted through the working channel of the endoscope, and which utilizes energy to penetrate through the tissue. A guide wire is then fed through the endoscope and is passed through the puncture in the stomach wall and into the peritoneal cavity. The needle knife is removed, leaving the guide wire as a placeholder. A balloon catheter is then passed over the guide wire and through the working channel of the endoscope to position the balloon within the opening in the stomach wall. The balloon can then be inflated to increase the size of the opening, thereby enabling the endoscope to push against the rear of the balloon and to be fed through the opening and into the peritoneal cavity. Once the endoscope is positioned within the peritoneal cavity, numerous procedures can be performed through the working channels of the endoscope.

While the current methods and devices used to penetrate tissue are effective, one drawback is the risk of damaging adjacent organs and tissue as the needle is pushed through the tissue. Due to the low amount of energy and force of penetration needed to pass through tissue, there is the risk of penetrating adjacent tissue that is intended to be left unharmed during the procedure. For example, various tissue puncturing devices are disclosed in U.S. Patent Publication No. US 2007/0155306 A1, entitled “Flexible Endoscopic Safety Needle”, the disclosure of which is herein incorporated by reference in its entirety. While such devices have greatly reduced the risk of inadvertently penetrating adjacent tissues and/or organs, due to the low amount of energy and force of penetration needed to pass through tissues, some risk remains of penetrating adjacent tissue that is intended to be left unharmed during the procedure. Accordingly, there remains a need for improved devices for forming a passage through tissue while minimizing the risk of inadvertently damaging adjacent tissue and/or organs while forming the passage.

The foregoing discussion is intended only to illustrate some of the shortcomings present in the field of the invention at the time, and should not be taken as a disavowal of claim scope.

### SUMMARY

The present invention provides devices and methods for forming passages and holes through tissue. In one exemplary embodiment, a device for forming a passage in tissue is provided and includes a flexible elongate shaft that has a distal end and a proximal end. The distal end may terminate in a circumferentially extending tissue-cutting edge. A control member may interface with the proximal end of the elongate shaft to selectively impart a rotary motion thereto. In other embodiments, a stylet may be disposed within the distal end of the elongate shaft. The stylet can be positioned with the elongate shaft such that the blunt end thereof is slightly distal to the tissue-cutting edge to prevent tissue contact, and a proximal position in which the blunt end is in a proximal position relative to the tissue-cutting edge to allow the cutting edge to cut through tissue. The blunt end may be moveable from the distal position to the proximal position when the blunt end is advanced into a tissue surface. The device can also include a biasing element adapted to bias the blunt end to the distal position after the tissue-cutting edge has cut through the tissue and the resistance is relieved from the blunt end.

The device can further include an outer sheath that is disposed around at least a portion of the elongate shaft. The outer sheath can be movable relative to the elongate shaft and stylet to allow the elongate shaft and blunt end of the stylet to be fully contained within the outer sheath, for example, during insertion of the device through an endoscope. In other embodiments, the outer sheath can include an expandable member, for example, an expandable balloon, disposed around a portion thereof and adapted to selectively expand radially to increase a size of a passage cut by the tissue-cutting edge.

Also disclosed herein are methods for forming a passage through tissue. In one embodiment, the method can include inserting a flexible elongate shaft that has a distal end with circumferentially extending tissue-cutting edge formed thereon through a body lumen such that a proximal end portion of the flexible elongate shaft protrudes out of the body lumen. The method may further include contacting a target tissue within the body with the circumferentially extending cutting edge and applying a rotary motion to the proximal end portion of the flexible elongate shaft to cause the circumferentially extending tissue-cutting edge to cut through the target tissue.

In another embodiment, an expandable member can be positioned within a passage cut through the tissue by the tissue-cutting edge. The expandable member can optionally be formed on an outer sheath disposed around at least a portion of the elongate shaft, which can be expanded to increase a size of the passage. In an exemplary embodiment, the device can be inserted through an endoscope, and, after the expandable member is expanded, the endoscope can be advanced over the device and against the expandable member to push the expandable member and the endoscope through the expanded passage.

In another embodiment, the device can be inserted through a working channel of an endoscope. The blunt end of the stylet and the flexible elongate shaft can be fully contained within an outer sheath when the device is inserted through an endoscope. The outer sheath may be positioned to enable the blunt end of the stylet and flexible elongate shaft to be advanced distally beyond a distal end of the outer sheath prior to positioning the blunt end adjacent to a tissue surface to be penetrated.

These and other objects and advantages of the present invention shall be made apparent from the accompanying drawings and the description thereof.

### BRIEF DESCRIPTION OF THE FIGURES

The accompanying drawings, which are incorporated in and constitute a part of this specification, illustrate embodiments of the invention, and, together with the general description of the invention given above, and the detailed description of the embodiments given below, serve to explain various principles of the present invention.

FIG. 1 is a perspective view of one embodiment of a device for cutting a passage through tissue of the present invention with a dilating balloon thereof in an expanded condition;

FIG. 1A is another perspective view of the device of FIG. 1 with the dilating balloon thereof in a collapsed condition;

FIG. 2 is a top view of a portion of a handle assembly embodiment of the present invention;

FIG. 3 is a cross-sectional view of a portion of the handle assembly of FIG. 2 taken along line 3-3 in FIG. 2;

FIG. 4 is a cross-sectional view of a portion of the handle assembly of FIG. 2 with the sheath attachment portion in a distal extended position;

FIG. 5 is another cross-sectional view of a portion of the handle assembly with the sheath attachment portion in the proximal retracted position;

FIG. 6 is a cross-sectional view of a portion of the handle assembly of the present invention with the sheath attachment portion in the extended distal position;

FIG. 7 is a cross-sectional view of the handle assembly of FIG. 2 taken along line 7-7 in FIG. 2;

FIG. 8 is a perspective view of a locking button embodiment of the present invention;

FIG. 9 is a cross-sectional view of a distal end portion of a needle shaft embodiment of the present invention;

FIG. 10 is a cross-sectional view of a portion of another needle shaft embodiment of the present invention;

FIG. 11 is a top view of a handle assembly of the present invention with the sheath attachment portion in a distal extended position;

FIG. 12 is a cross-sectional view of the handle assembly of FIG. 11 taken along line 12-12 in FIG. 11;

FIG. 13 is a perspective view of a thumbwheel embodiment of the present invention;

FIG. 14 is a side view of the thumbwheel of FIG. 13;

FIG. 15 is a side view of a distal end portion of a stylet embodiment of the present invention;

FIG. 16 is a top view of a reset cap embodiment of the present invention;

FIG. 17 is a side view of a reset cap embodiment of the present invention;

FIG. 18 is a cross-sectional view of the reset cap of FIGS. 16 and 17 taken along line 18-18 in FIG. 16;

FIG. 19 is a side view of a locking knob embodiment of the present invention;

FIG. 20 is an enlarged partial cross-sectional view of a portion of a handle assembly embodiment of the present invention;

FIG. 21 is a side view of a pusher cam embodiment of the present invention;

FIG. 22 is a side view of an inside cam embodiment of the present invention;

FIG. 23 is a perspective view of the inside cam embodiment of FIG. 22;

FIG. 24 is an enlarged cross-sectional view of another portion of a handle assembly embodiment of the present invention;

FIG. 25 is a side view of an outside cam embodiment of the present invention;

FIG. 26 is a cross-sectional view of the outside cam of FIG. 25 taken along line 26-26 in FIG. 25;

FIG. 27 is a perspective view of the outside cam of FIGS. 25 and 26;

FIG. 28 is a schematic view of a patient that illustrates methods of using various embodiments of the present invention;

FIG. 29 is a partial cross-sectional view of a distal end of an overtube positioned adjacent a portion of the abdominal wall, with a guide wire passed therethrough;

FIG. 30 illustrates the application of vacuum through the overtube to the portion of abdominal wall depicted in FIG. 29;

FIG. 31 illustrates contacting the abdominal wall with the stylet and needle assemblies of the present invention;

FIG. 32 illustrates cutting through the abdominal wall with the needle assembly of the present invention; and

FIG. 33 illustrates the position of the distal end of the needle assembly and the stylet after the needle assembly has cut a hole through the abdominal wall.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the various embodiments of the present invention is defined solely by the claims. The features illustrated or described in connection with one exemplary embodiment may be combined with the features of other embodiments. Such modifications and variations are intended to be included within the scope of the present invention.

Various exemplary methods and devices are provided for forming a passageway through tissue. In various embodiments, the device may comprise a flexible elongate shaft that has a distal end that has a circumferentially extending tissue-cutting edge formed thereon. The device may further include a control member configured to interface with the proximal end of the elongate shaft to selectively impart a rotary motion thereto. As the distal end of the elongate shaft is brought into contact with the target tissue and then rotated, the circumferentially extending tissue-cutting edge cuts a hole through the tissue. In other embodiments, the circumferentially extending tissue-cutting edge may be formed on a separate insert that is otherwise attached to the distal end of the elongate shaft. A stylet may also be provided within the elongate shaft and be selectively adjustable such that a blunt distal end of the stylet may oriented to protrude slightly beyond the tissue-cutting edge. Upon contact with the target tissue, the blunt edge of the stylet is pushed into the hollow distal end of the elongate shaft to expose the circumferentially extending tissue-cutting edge to the tissue. The stylet may also interface with a biasing arrangement in the handle assembly such that after the tissue-cutting edge has passed through the tissue and the resistance applied to the blunt end by the tissue is relieved, the blunt end is automatically biased to its starting position to thereby protect the tissue-cutting edge from inadvertent contact with adjacent tissue and/or organs. In various embodiments, for example, the stylet may function as a guide wire after the entry hole has been cut through the tissue. Also in various embodiments, the needle assembly and stylet may be supported within an outer sheath that may be selectively moved from a distal position wherein the distal end portion of the needle assembly and blunt end of the stylet are completely received within the outer sheath and a proximal position wherein the distal end portion and blunt end protrude out of the distal end of the outer sheath. A locking assembly may be provided in the handle assembly to selectively lock the outer sheath in the distal and proximal positions. While the device can be used in a variety of applications, it is preferably used in endoscopic or laparoscopic surgery. For example, the device can be inserted translumenally, and then penetrated through a tissue surface, such as the stomach or colon, to form a passage or hole through the tissue to provide access to other areas of the body, such as the abdominal cavity.

It will be appreciated that the terms “proximal” and “distal” are used herein with reference to a clinician manipulating the handle assembly 20 of the device 10 that protrudes out of the natural orifice. The term “proximal” referring to the portion closest to the clinician and the term “distal” referring to the portion located away from the clinician. It will be further appreciated that for convenience and clarity, spatial terms such as “vertical”, “horizontal”, “up” and “down” may be used herein with respect to the drawings. However, surgical instruments are used in many orientations and positions, and these terms are not intended to be limiting and absolute.

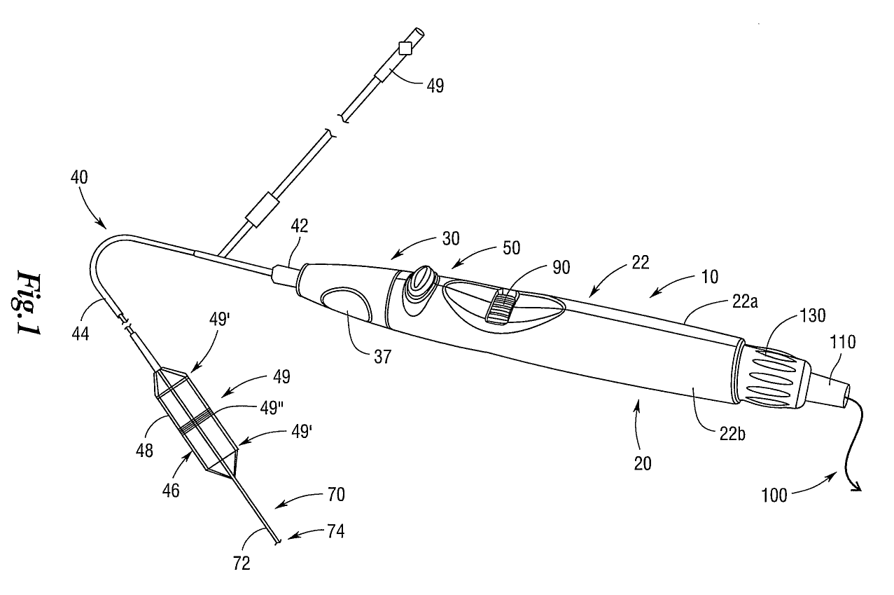

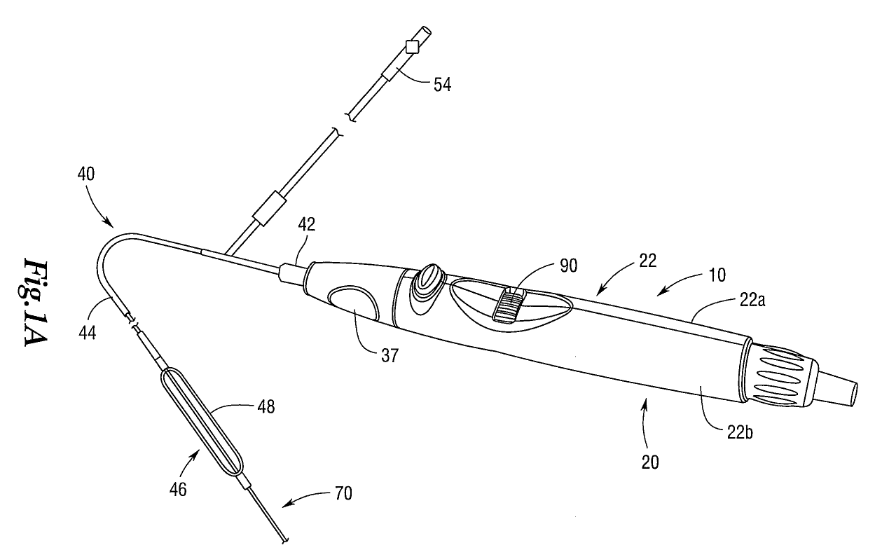

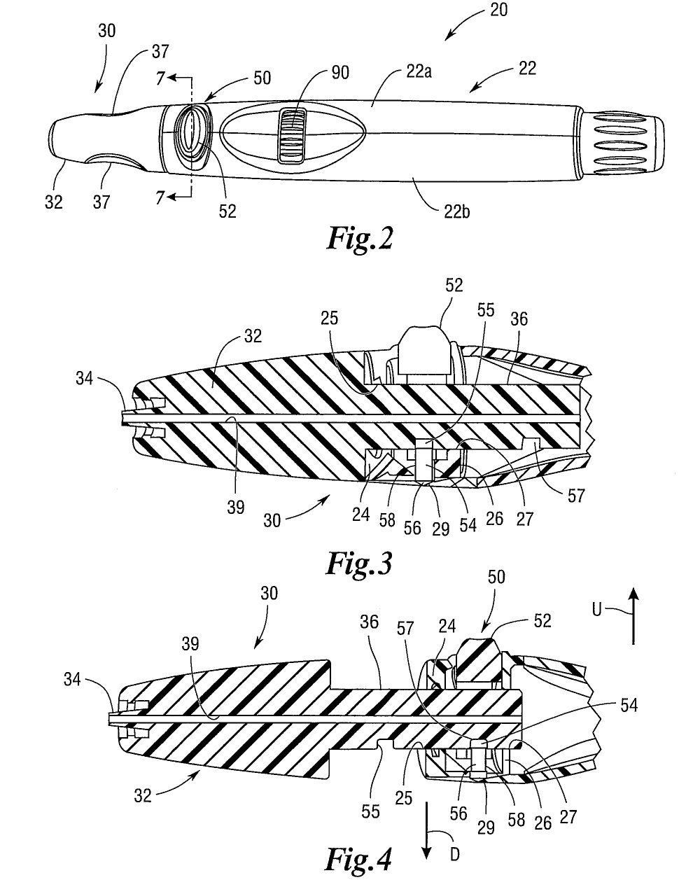

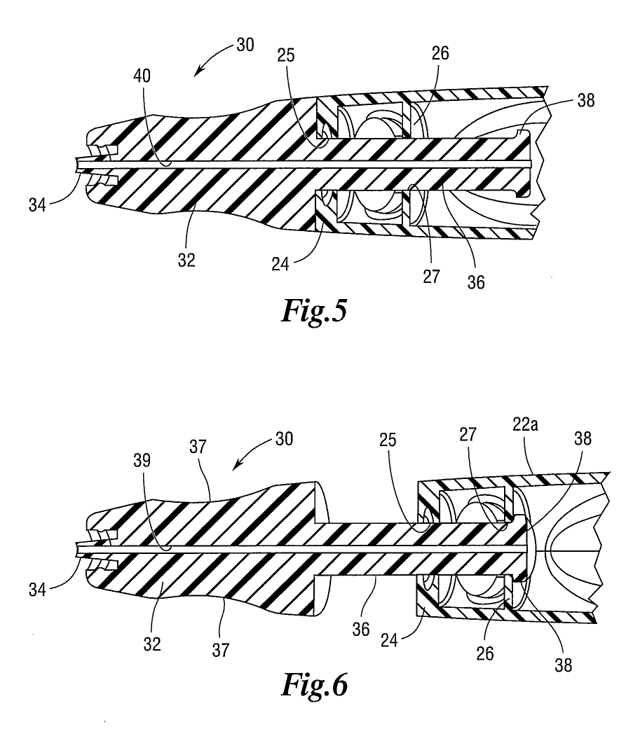

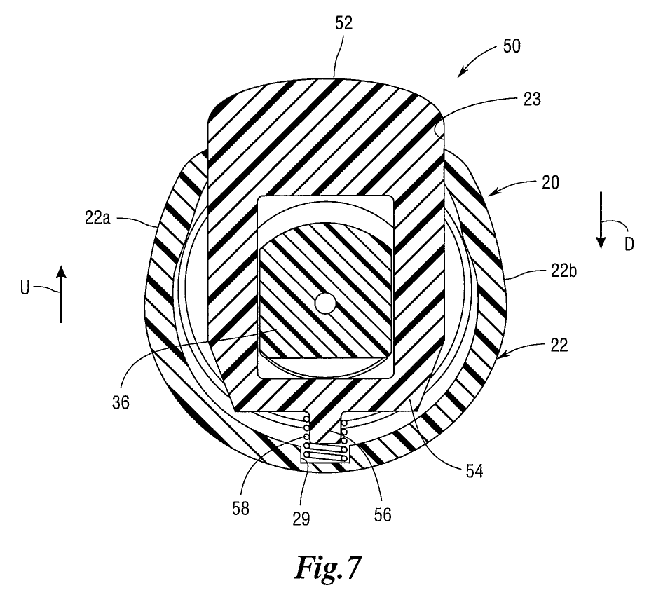

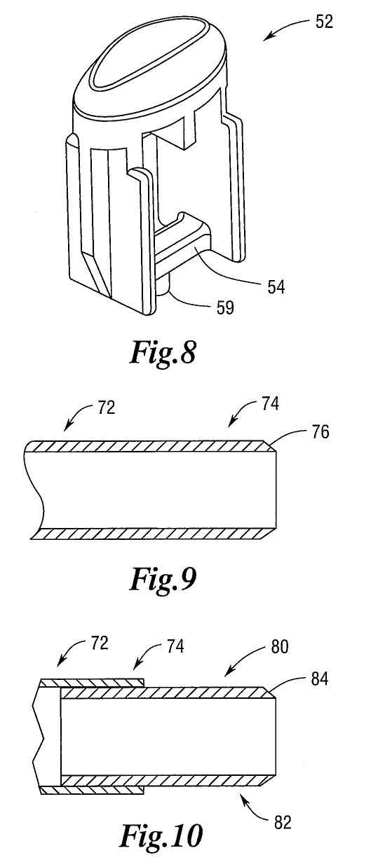

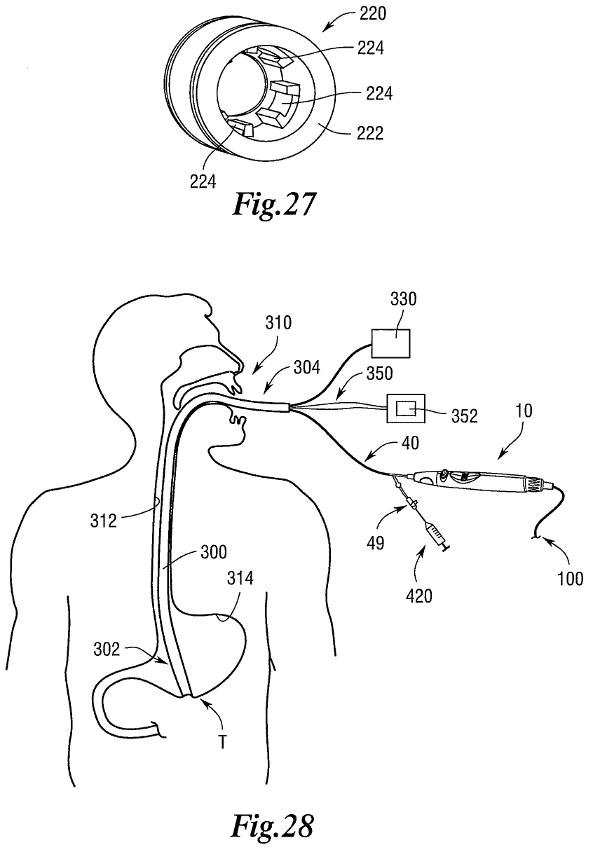

FIGS. 1-27 illustrate an exemplary embodiment of a device for forming a passageway through tissue. As shown, the device 10 generally includes a handle assembly 20 that may include a main body portion 22 that has a sheath attachment portion 30 movably coupled thereto. The handle assembly 20 of the device 10 can have any shape and size, but it is preferably adapted to facilitate grasping and manipulation of the device 10. In the illustrated embodiment, as shown in FIGS. 1 and 2, the handle assembly 20 has an elongate cylindrical configuration. The main body portion 22 of the handle assembly 20 can be formed from multiple pieces, or it can have a unitary configuration. In the illustrated embodiment, the main body portion 22 of the handle assembly 20 includes two halves 22a, 22b that mate together and that house the proximal portions of a needle assembly 70. The main body portion 22 further movably supports a sheath attachment portion 30 thereon that is coupled to an outer sheath 40.

As can be seen in FIGS. 3 and 4, the sheath attachment portion 30 has a distal end portion 32 that may have a luer-type connector fitting 34 formed thereon to enable a sheath fitting 42 (FIG. 1) to be attached thereto. A locking bar 36 protrudes proximally from the distal end portion 32 and is configured to be slidably received within passages 25 and 27 formed in inwardly extending flanges 24 and 26, respectively in the main body portion 22 of the handle assembly 20. Retaining protrusions 38 may be formed on the proximal end of the locking bar 36 to prevent the sheath attachment portion 30 from being detached from the main body portion 22 of the handle assembly 20. An axial passage 39 extends through the sheath attachment portion 30 to enable a needle assembly 70 to rotatably pass therethrough. A pair of opposed recessed areas or scalloped areas 37 may be provided in the outer surface of the distal end portion 32 to enable the surgeon to grasp the sheath attachment portion 30 between his or her thumb and index finger to facilitate axial movement of the sheath attachment portion 30 between a proximal position (FIGS. 1-3) and an the extended distal position (FIGS. 4 and 6).

The device 10 may be further provided with a locking assembly 50 for selectively locking the sheath attachment portion 30 in the distal and proximal positions. More particularly and with reference to FIGS. 3, 4, 7, and 8, the locking assembly 50 may include a locking button 52 that is movably supported within an opening 23 formed in the main body portion 22 of the handle assembly 20 for selective vertical movement in the up direction (arrow “U” in FIG. 7) and the down direction (arrow “D” in FIG. 7). The locking button 52 may further have a lock bar portion 54 that has a pin 56 protruding therefrom that may protrude into a corresponding cavity 29 formed in the main body portion 22 of the handle assembly 20. In addition, the button 53 may be formed with laterally protruding slide bars 53 that are received within slide grooves or recesses (not shown) formed in the main body portion 22 of the handle assembly 20. A coil spring 58 may extend into the cavity 29 and be journaled around the pin 56 to bias the locking button 52 in the “U” direction. See FIG. 7. As can be seen in FIGS. 3 and 4, the locking bar 36 may have a distal locking cavity 57 and a proximal locking cavity 59 formed therein for receiving the lock bar portion 54 therein. Thus, when the lock bar portion 54 is received within the distal locking cavity 55 (FIG. 3), the sheath attachment portion 30 is retained in the retracted proximal position and when the lock bar portion 54 is received in the proximal locking cavity 57 (FIG. 4), the sheath attachment portion 30 is locked in the extended distal position. Other methods of movably attaching the sheath attachment portion to the main portion 22 of the handle assembly 20 may be employed. For example, the locking bar portion (or other portion) of the sheath attachment portion may be threadedly coupled to the distal end of the main portion of the handle assembly such that the surgeon may advance or retract the sheath attachment portion simply by rotating the sheath attachment portion relative to the main body portion of the handle assembly.

The device 10 may further include an outer sheath assembly 40 that may include a sheath fitting 42 configured for attachment to the luer-type fitting 34 on the distal end portion 32 of the sheath attachment portion 30. The outer sheath assembly 40 may further include an outer sheath 44 that can be flexible or rigid, but in an exemplary embodiment, a distal end of the device 10 is adapted to be inserted translumenally, and therefore the outer sheath 44 can be semi-flexible or flexible to allow insertion through a tortuous lumen. The length of the outer sheath 44 can vary depending on the intended use of the device 10, but in an illustrated embodiment, the outer sheath 44 has an elongate length that is adapted for use translumenally. A person skilled in the art will appreciate that the outer sheath 44 is not a necessary component for the device 10 to cut through tissue and can be omitted.

In the illustrated embodiment, the sheath assembly 40 further includes an expandable member 46 that is adapted to increase the size of the passage formed in tissue by the tissue-cutting tip of the device. As shown, the expandable member 46 is in the form of a dilating balloon 48 that is configured to be inflated to expand the size of the passage cut by the tissue-cutting edge formed on the distal end of the needle. A person skilled in the art will further appreciate that a variety of other expandable members can be used to expand a through hole created by the tissue-cutting edge. The balloon 48 can be disposed at various locations, but FIG. 1 illustrates the balloon 48 disposed on the outer sheath 44. An inflation port 49 is provided in the sheath assembly 40 to enable the balloon 48 to be inflated using, for example, fluid or air introduced through an inflation lumen formed in and extending along the outer sheath 44. A person skilled in the art will also appreciate that any inflation lumen can be used to inflate the balloon 48, including a lumen internal or external to the outer sheath 44. In use, after the distal end of the needle has cut through the tissue, the device 10 can be advanced to position the deflated balloon 48, (FIG. 1A), within the hole. The balloon 48 may then be inflated by inserting a needle of a syringe to inject a saline solution or air therein to inflate the balloon 48 as will be discussed in further detail below to increase the size of the hole.

In various embodiments, the balloon 48 may be provided with reference indicia 49 thereon that may comprise a plurality of lines or markings on the balloon 48 to assist the surgeon in ascertaining the position of the balloon 48 relative to the tissue. In the embodiment depicted in FIG. 1, for example, a relatively “thin” line 49′ is provided adjacent the distal end and the proximal end of the balloon 48 and a relatively thicker line 49″ is provided in the center area of the balloon 48. However, other numbers and types of markings may be employed.

The device 10 also includes a needle assembly 70 that may comprise an elongate shaft in the form of a needle shaft 72 that may be fabricated from, for example, NITINOL which comprises a nickel titanium that is superelastic and able to withstand a small amount of deformation when a load is applied and return to its original shape when the load is removed. However, the needle shaft 72 may also be fabricated from other suitable materials without departing from the spirit and scope of the present invention. For example, one skilled in the art will appreciate that the needle assembly 70 can be made from a variety of biocompatible materials that have properties sufficient to enable portions of the needle shaft 72 extending from the handle assembly 20 to be inserted and moved within channels of a body lumen. The needle shaft 72 can also have a length that can vary depending on the intended use of the device, but in an exemplary embodiment, the length is adapted for use translumenally. A diameter of the needle shaft 72 can also vary, but the diameter is preferably sufficient to slidably receive the stylet assembly 100. In various embodiments, for example, the needle shaft 72 may have an outer diameter of approximately 0.032 inches (0.8 mm).

The needle shaft has a distal end 74. As can be seen in FIG. 9. In various embodiments, the distal end 74 has a tissue-cutting edge 76 formed around the outer circumference of the distal end thereof that will cut through tissue as it is rotatably advanced into the tissue. In the illustrated embodiment, the tissue-cutting edge 76 comprises a sharpened edge that is formed on the outside diameter of the distal end 74. Other embodiments may comprise a sharpened end that is formed on the inside diameter. Still other embodiments may comprise an abrasive edge, a serrated edge, etc. In the embodiment depicted in FIG. 10, an insert 80 is attached to the distal end 74 of the needle shaft 72. The tissue-cutting edge 84 is formed around the circumference of the distal end 82 of the insert 80. As will be discussed in further detail below, as the tissue cutting edge 76/84 is rotatable advanced into the tissue, the tissue-cutting edge 76/84 penetrates and cuts through the tissue with little axial force applied thereto.

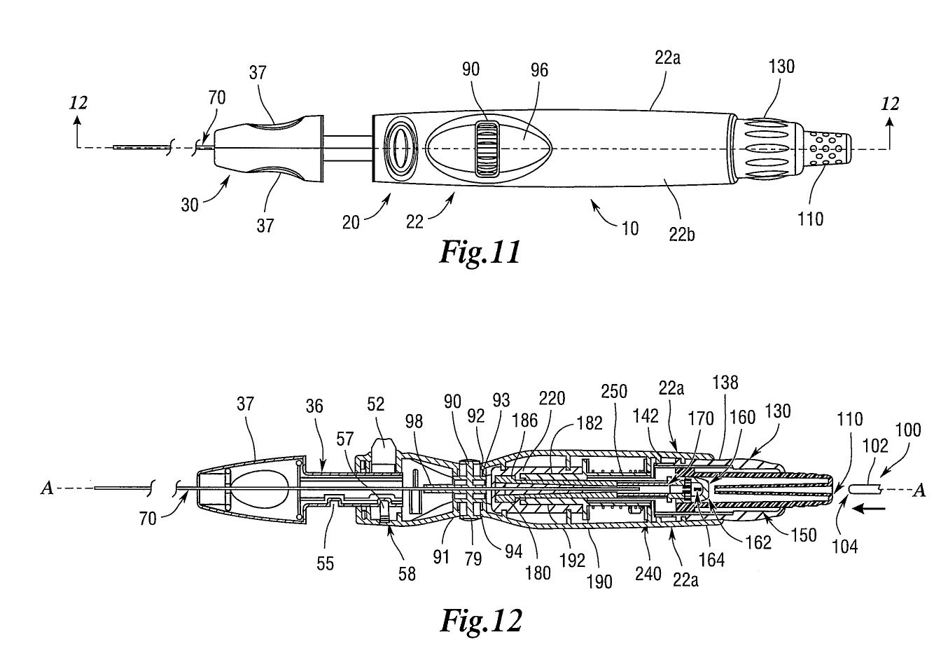

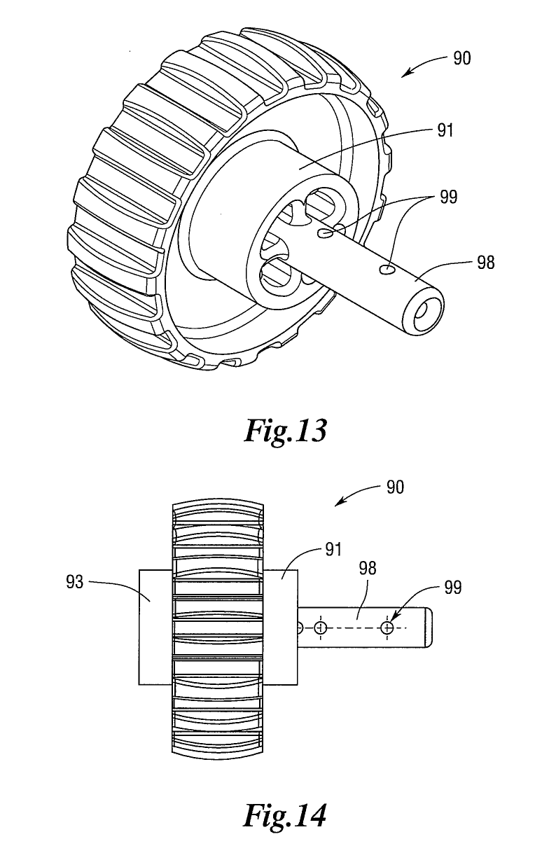

As can be seen in FIG. 12, the proximal end 79 of the needle shaft is non-movably coupled to a thumb wheel 90 that is rotatably supported within the main body portion 22 of the handle assembly 20. The thumbwheel 90 protrudes through two opposed openings 92 and 94 in the main body portion 22 to enable the surgeon to readily rotate the thumbwheel 90 about the device's axis A-A. As can be seen in FIG. 11, the main body portion may be provided with recesses or scalloped portions 96 (an opposing scalloped portion 96 is no shown in FIG. 11) to enable the surgeon to rotate the thumb wheel 90 between his or her thumb and index finger, for example. As can be seen in FIG. 14, the thumbwheel 90 has a pair of hubs 91 and 93 that are adapted to be rotatably supported within the main body portion 22 of the handle assembly 20. As can also be seen in FIGS. 13 and 14, the thumbwheel 90 includes a hollow stem portion 98 adapted to receive the needle shaft 72 therein. The proximal portion 79 of the needle shaft 72 is non-rotatably affixed to the hollow stem portion 98 such that when the thumbwheel 90 is rotated about axis A-A, the needle shaft 72 also rotates. In one embodiment, the proximal portion 79 of the needle shaft 72 may be pinned to the stem portion 98 by inserting pins (not shown) through holes 99 in the stem portion 98 and then glued with a biocompatible adhesive. However, other methods of non-rotatably affixing the proximal portion of the needle shaft 72 to the thumbwheel 90 may be employed.

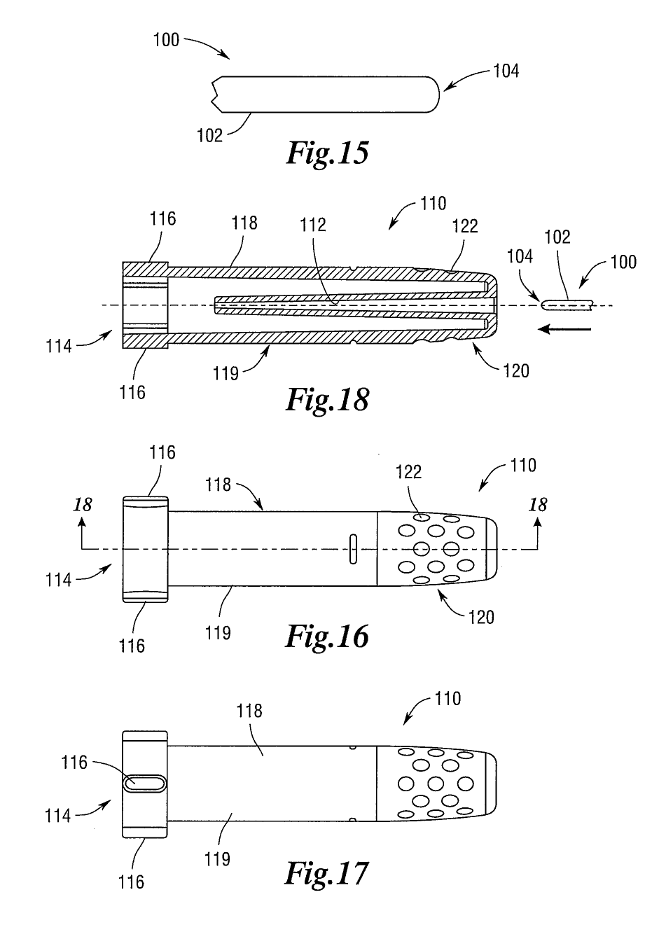

The device 10 may also include a stylet assembly 100 that may slidably extend through the handle assembly 20 and the needle assembly 70 to protect the circumferentially extending tissue-cutting edge 76 until the distal end 74 of the needle shaft 72 is positioned against the tissue to be penetrated. As can be seen in FIG. 15, the stylet has a distal end 104 that is substantially blunted or rounded for preventing the tissue-cutting edge 76 on the distal end 74 of the needle shaft 72 from inadvertently damaging adjacent tissue or organs. As will be explained in further detail below, the blunted distal end 104 of the stylet 102 can be positioned relative to the tissue-cutting edge 76 of the needle shaft 72 to essentially render the distal end 74 of the needle shaft 72 blunt and prevent it from penetrating or cutting tissue. The distal end 104 of the stylet 102 can be moved proximally within the distal end 74 of the needle shaft 72 to expose the circumferentially extending tissue-cutting edge 76 to cut through tissue as it is rotated. Once the distal end 74 of the needle shaft 72 penetrates through tissue, the distal end 104 of the stylet 102 can return to its initial, distal position to protect the tissue-cutting edge 76 to prevent unintentional cutting and/or puncture of adjacent tissue.

The adjustment and operation of the stylet assembly 100 can be understood from reference to FIGS. 12-27. In the illustrated embodiment, the stylet 102 extends through a passage 112 in a reset button 110. See FIG. 18. The reset button 110 has a distal end 114 that has a pair of diametrically opposed ears 116 protruding therefrom that are configured to be slidably received within a lock knob 130 as will be discussed in further detail below. In addition, the reset button 110 may have a shaft portion 118 that has a relatively smooth outer surface 119. The shaft portion 118 terminates in a proximal end 120 that may be provided with dimples 122 to provide an enhanced gripping surface.

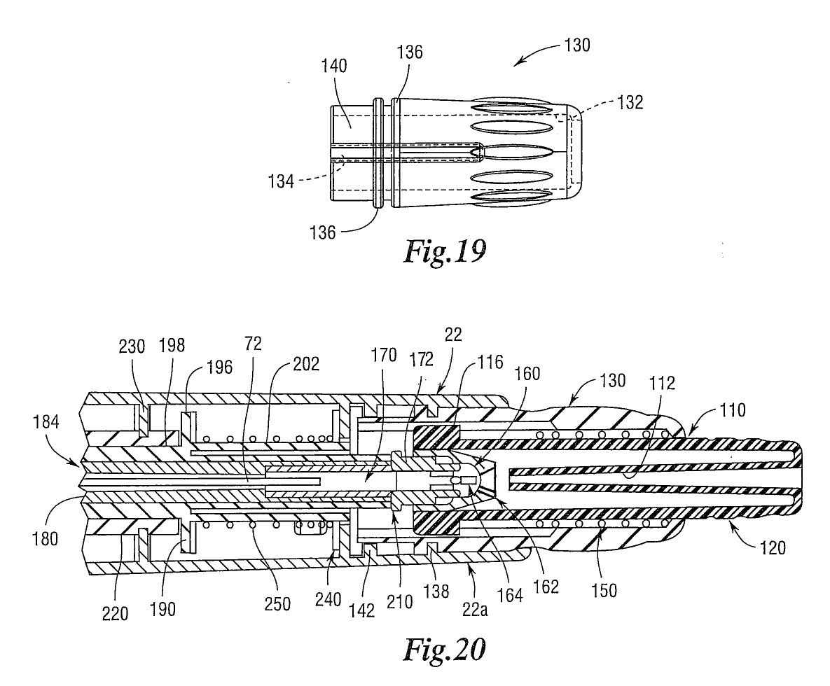

As indicated above, the reset button 110 is adapted to be slidably received within a lock knob 130 that may be configured as shown in FIG. 19. As can be seen in that Figure, the lock knob 130 may have an axial passage 132 therethrough into which the reset button 110 is received as illustrated in FIG. 12. In addition, the lock knob 130 has two diametrically opposed elongated recesses 134 that correspond to the ears 116 on the reset button 110. Thus, the reset button may slide axially relative to the lock knob 130, but rotates therewith. The lock knob 130 may have a pair of spaced, circumferentially extending, flanges 136 formed thereon to receive an inwardly extending flange 138 formed on the main body portion 22 of the handle assembly 20 as shown in FIGS. 12 and 20. Those of ordinary skill in the art will understand that such arrangement serves to rotatably affix the lock knob 130 to the main body portion 22 of the handle assembly. In addition, the lock knob 130 may have a distal hub portion 140 that is rotatably supported within the main body portion 22 of the handle assembly 20 by inwardly extending flange 142. As can also be seen in FIGS. 12 and 20, a stylet spring 150 may be provided within the lock knob 130 to bias the reset button 110 in the distal direction “D-D”.

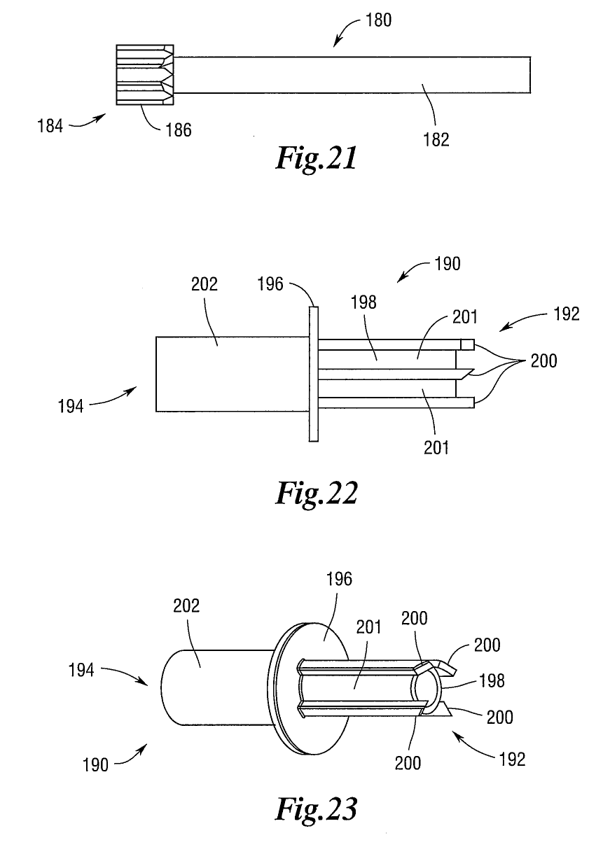

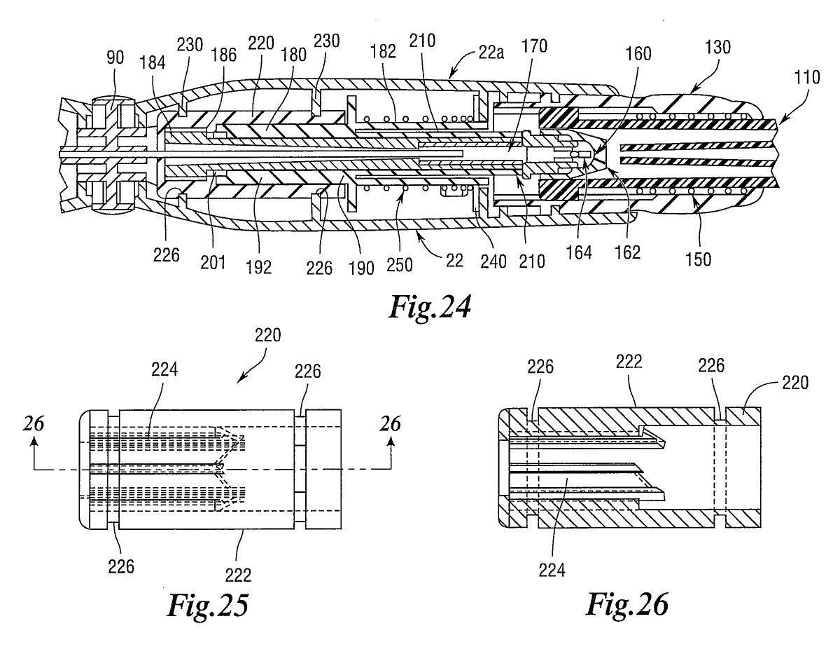

Supported within the distal end 116 of the reset button 110 is a collet assembly 160. See FIGS. 12 and 20. In various embodiments, the collet assembly 160 may include a collet cap 162 and a collet body 164 through which the stylet 102 may pass. The collet body 164 is received in the proximal end portion 172 of a pusher cam 170 as can be seen in FIGS. 12 and 20. As can be seen in FIG. 20, the pusher cam 170 has a proximal end 172 that is configured to interface with the collet body 164 and a hollow shaft portion 174 that protrudes into a pusher cam 180. As can be seen in FIG. 21, the pusher cam 180 has a shaft portion 182 that has a hollow passage 184 (FIG. 20) therethrough for receiving the shaft 174 of pusher cam 170 and the proximal end portion of needle shaft 72. The distal end 184 of the pusher cam 180 has a series of pointed gear teeth 186 formed around its circumference as shown. Journaled on the shaft 182 of the pusher cam 180 is an inside cam 190 that has a distal end 192 and a proximal end 194 that is separated by a flange 196. The distal end 192 of the inside cam 190 has a shaft portion 198 that has a plurality of elongated pointed teeth 200 formed thereon that define grooves 201 therebetween. The proximal end 194 of the inside cam 190 comprises a hub 202. As can be seen in FIGS. 12 and 24, a retainer tube 210 supports the shaft portion 182 of the pusher cam 180 within the hub portion 202 of the inside cam 190.

As can be seen in FIGS. 12 and 24, the distal end portion 192 of the inside cam 190 is movably received within an outside cam 220. An embodiment of the outside cam 220 is depicted in FIGS. 24-26. As can be seen in those Figures, the outside cam 220 comprises a hollow body portion 222 that has a series of teeth receptacles 212 formed therein for receiving the teeth 186 of the pusher cam 180 therein. A pair of circumferentially extending grooves 226 are provide therein for receiving corresponding inwardly extending flanges 230 formed on the main body portion 22 of the handle assembly 20. See FIG. 24. As can also be seen in FIG. 24, a slip washer 240 and a spring 250 are journaled on the hub portion 202 of the inside cam 190.

The stylet cam mechanism functions as follows. The retainer tube 210 is placed over the pusher cam 180 and acts as a hard stop to allow the pusher cam 180 and inside cam 190 to disengage. In the retracted position, the grooves 201 on the inside cam 190 are misaligned with the teeth 186 on the pusher cam 180 such that the inside cam 190 cannot extend fully into the outside cam 220. When the stylet 102 is retracted (moved in the proximal direction), the inside cam 190 is rotated, assisted by the cam spring to allow the inside cam grooves 201 and pusher cam teeth 186 to align. The inside cam 190 can now fully extend into the outside cam 220 assisted by a pusher spring.

FIG. 28 schematically illustrates the distal end 302 of an overtube 300 that has been inserted through a patient's mouth 310 and esophagus 312 into the stomach 314 to perform various surgical procedures. The overtube 300 may be formed of a highly flexible biocompatible material that slides through the digestive tract or other body passage with a minimum of frictional resistance. The overtube 300 has an elongated, generally tubular configuration with a proximal end 304 located externally to the patient's mouth 310. The overtube 300 serves to provide a repeatable tubular passageway through which an endoscope 350 as well as the tissue penetration devices of various embodiments of the present invention may pass. Such overtubes are known in the art and may have distal end portions that are selectively reconfigurable or steerable. For example, pending U.S. patent application Ser. No. 11/756,914, filed Jun. 1, 2007 and entitled “Integrated Securement and Closure Apparatus”, the disclosure of which is herein incorporated by reference in its entirety may be employed. Other known overtube arrangements may also be employed.

In the specific illustration of FIG. 28, the overtube 300 has accessed the patient's stomach 314 through the patient's mouth 310. Depending upon the location of the specific portion of the body one which a diagnostic or therapeutic intervention is desired, access to the location may be made through alternative paths. For example, for a surgical intervention in the lower colon, access through the patient's anus may be preferable. Furthermore, in some applications, it may be possible to access the targeted tissue without the necessity of extending the overtube 300 through the digestive tract.

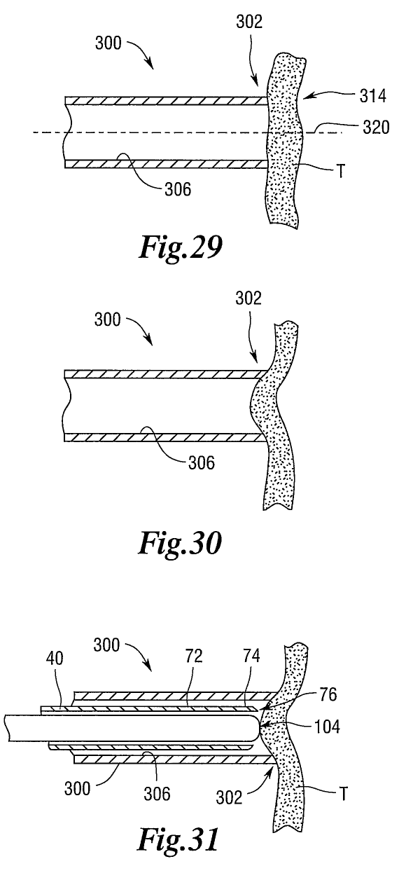

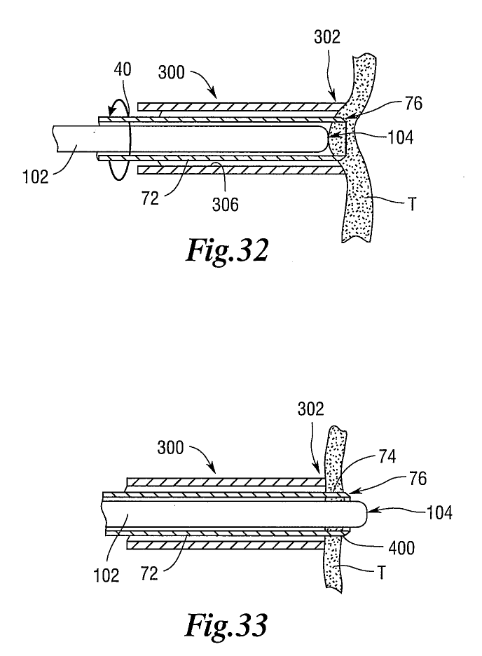

The overtube 300 includes one or more centrally disposed tool-receiving passages that extend continuously from the proximal end to the distal end to provide one or more working channels through which a surgeon can slide endoscopic tools. As can be seen in FIG. 29, the distal end 302 of the overtube 300 is shown after it has been manipulated from a location external to the patient so as to engage the distal end 302 of the overtube against a targeted tissue “T” of the stomach 314 as is known in the art. Depending upon the flexibility and ease with which the specific type of overtube used can be manipulated, it may be desirable to first penetrate the target tissue “T” with a guide wire 320, and then use the guide wire 320 to sliding direct the overtube 300 to the target location on the stomach wall or other desired tissue. The endoscope 350 commonly accommodates a camera that provides the surgeon with the ability to monitor the surgical site through a video screen 352 in the surgical suite as is known. When moved to this position, a vacuum is applied to the passage of the overtube 300 to sealingly engage the distal end 302 of the overtube 300 against the organ wall which, in the illustrated embodiment, is the tissue “T”. The source 330 for the vacuum might comprise a vacuum wall port in a typical operating room, or any other suitable source. In any event the source of vacuum is in fluid communication with the passage 306.

In the illustrated embodiment, the application of vacuum draws a portion of the stomach wall “T” slightly into the passage and may also serve to slightly draw that portion of stomach wall away from any organ or tissue immediately adjacent thereto. See FIG. 30. Thereafter, the guide wire 320 may be withdrawn (if used) and then the distal end 76 of the needle shaft 72 inserted through the overtube 300. Prior to inserting, however, the surgeon would have adjusted the stylet 102 such that the blunt end 104 thereof protrudes slightly out of the distal end 74 of the needle shaft 72 so as to protect the circumferentially-extending tissue-cutting edge 76 thereon. This can be done by rotating the locking knob 130 to release the collet assembly 160 to enable the surgeon to slide the stylet 102 to the desired position. Initially for example, the blunt end 104 of the stylet 102 may be adjusted to protrude slightly distally beyond the circumferentially extending tissue-cutting edge 76. FIG. 31 illustrates the position of the distal end 74 of the needle shaft 72 and blunt end 104 just as the blunt end 104 of the stylet 102 contacts the tissue “T”.

After the blunt end 104 of the stylet 102 contacts the tissue “T”, the stylet is biased inwardly into the distal end 74 of the needle shaft 72 to expose the tissue-cutting edge 76. The surgeon then applies a rotary motion to the needle shaft 72 by rotating the thumbwheel 90 relative to the main body portion 22 of the handle assembly 20. When the cutting edge 76 has cut through the tissue “T”, the resistance applied to the stylet 102 is relieved and the stylet 102 immediately is biased to it original preset position wherein the blunt end 104 protrudes slightly beyond the tissue-cutting edge 76 to prevent the tissue-cutting edge 76 from inadvertently damaging adjacent tissue or organs. See FIG. 34.

After the hole 400 has been cut through the tissue “T”, the surgeon may then advance the dilating balloon 48 into the hole 400 and inflate it with air or saline solution in a known manner with a syringe 420 to expand the hole 400. Once the hole has been expanded the device 10 can be removed. However, the stylet 102 may remain and function as a guide wire to enable an endoscope or other instrument to be inserted through the hole. In another exemplary embodiment, an endoscope 350 can be passed through the esophagus and positioned within the stomach, and a tissue-penetrating device 10, can be introduced through a working channel of the endoscope 350 and used to cut a passage hole in the stomach wall. Once the balloon 46 has been inflated and the size of the through hole in the stomach wall has been increased, the endoscope can be advanced into the expandable member to push the expandable member and the endoscope through the hole and into the abdominal cavity. Additional instruments and devices can then be passed through the working channel of the endoscope to perform various procedures.

One skilled in the art will appreciate further features and advantages of the invention based on the above-described embodiments. Accordingly, the invention is not to be limited by what has been particularly shown and described, except as indicated by the appended claims.

While the present invention has been illustrated by description of several embodiments and while the illustrative embodiments have been described in considerable detail, it is not the intention of the applicant to restrict or in any way limit the scope of the appended claims to such detail. Additional advantages and modifications may readily appear to those skilled in the art. Those of ordinary skill in the art will readily appreciate the different advantages provided by these various embodiments. While several embodiments of the invention have been described, it should be apparent, however, that various modifications, alterations and adaptations to those embodiments may occur to persons skilled in the art with the attainment of some or all of the advantages of the invention. For example, according to various embodiments, a single component may be replaced by multiple components, and multiple components may be replaced by a single component, to perform a given function or functions. This application is therefore intended to cover all such modifications, alterations and adaptations without departing from the scope and spirit of the disclosed invention as defined by the appended claims.

The devices disclosed herein can be designed to be disposed of after a single use, or they can be designed to be used multiple times. In either case, however, the device can be reconditioned for reuse after at least one use. Reconditioning can include an combination of the steps of disassembly of the device, followed by cleaning or replacement of particular pieces, and subsequent reassembly. In particular, the device can be disassembled, and any number of particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those of ordinary skill in the art will appreciate that the reconditioning of a device can utilize a variety of different techniques for disassembly, cleaning/replacement, and reassembly. Use of such techniques, and the resulting reconditioned device, are all within the scope of the present application.

Preferably, the invention described herein will be processed before surgery. First a new or used instrument is obtained and, if necessary, cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or higher energy electrons. The radiation kills bacteria on the instrument and in the container. The sterilized instrument can then be stored in the sterile container. The sealed container keeps the instrument sterile until it is opened in the medical facility.

Any patent, publication, or other disclosure material, in whole or in part, that is said to be incorporated by reference herein is incorporated herein in its entirety, but only to the extent that the incorporated materials do not conflict with existing definitions, statements, or other disclosure material specifically set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The invention which is intended to be protected is not to be construed as limited to the particular embodiments disclosed. The embodiments are therefore to be regarded as illustrative rather than restrictive. Variations and changes may be made by others without departing from the spirit of the present invention. Accordingly, it is expressly intended that all such equivalents, variations and changes which fall within the spirit and scope of the present invention as defined in the claims be embraced thereby.

What is claimed is:

1. A device for forming a passage in tissue, comprising:
a substantially flexible elongate shaft having distal end and a proximal end, said distal end terminating in a circumferentially extending sharpened tissue-cutting edge; and
a control member interfacing with said proximal end of said elongate shaft to selectively impart a rotary motion thereto.

2. The device of claim 1 wherein said elongate shaft is hollow and wherein said device further comprises a stylet disposed within the distal end of said hollow elongate shaft and is movable relative to the tissue-cutting edge between a distal position in which a blunt end of the stylet is distal of the tissue-cutting edge to prevent tissue contact, and a proximal position in which the blunt end is proximal of the tissue-cutting edge to expose the tissue-cutting edge to tissue.

3. The device of claim 2, wherein the blunt end of the stylet is adapted to move from the distal position to the proximal position when the blunt end is advanced into a tissue surface.

4. The device of claim 2, further comprising a biasing assembly configured to bias the blunt end of the stylet to the distal position.

5. The device of claim 2, further comprising an outer sheath disposed around at least a portion of the elongate shaft.

6. The device of claim 5, wherein the outer sheath is selectively movable relative to the elongate shaft.

7. The device of claim 6 wherein said outer sheath may be selectively locked in at least two different axial positions relative to said elongate shaft.

8. The device of claim 5, wherein the outer sheath includes an expandable member disposed around a portion thereof and adapted to expand radially.

9. The device of claim 7, wherein the expandable member comprises an expandable balloon.

10. The device of claim 8 further comprising at least one reference indicia on said expandable member.

11. A method for processing an instrument for surgery, the method comprising:
obtaining the device of claim 1;
sterilizing the device; and
storing the device in a sterile container.

12. A device for forming a passage in tissue, comprising:
a handle assembly;
a substantially flexible elongate shaft having distal end and a proximal end portion, said distal end terminating in a circumferentially extending sharpened tissue-cutting edge; and
a thumbwheel rotatably supported by said handle assembly and coupled to said proximal end portion of said elongate shaft to enable a rotary motion to be applied thereto.

13. The device of claim 12 further comprising an outer sheath disposed around at least a portion of the elongate shaft and being selectively axially movable relative thereto.

14. The device of claim 13 wherein said handle assembly comprises a main body portion and wherein said outer sheath is coupled to a sheath attachment member that is movably coupled to said main body portion.

15. The device of claim 14 wherein said sheath attachment member is selectively lockable in at least two axial positions relative to said main body portion of said handle assembly.

16. The device of claim 12 wherein said elongate shaft is hollow and wherein said device further comprises a stylet disposed within the distal end of said hollow elongate shaft and is movable relative to the tissue-cutting edge between a distal position in which a blunt end of said stylet is distal of the tissue-cutting edge to prevent tissue contact therewith, and a proximal position in which the blunt end is proximal of the tissue-cutting edge to allow the tissue-cutting edge to contact tissue.

17. The device of claim 16, wherein the blunt end is adapted to move from the distal position to the proximal position when the blunt end is advanced into a tissue surface.

18. A method for forming a passage through tissue, comprising:
inserting a flexible elongate shaft that has a distal end with circumferentially extending sharpened tissue-cutting edge formed thereon through a body lumen such that a proximal end portion of the flexible elongate shaft protrudes out of the body lumen;
contacting a target tissue within the body with the circumferentially extending tissue-cutting edge; and
applying a rotary motion to the proximal end portion of the flexible elongate shaft to cause the circumferential tissue-cutting edge to cut through the target tissue.

19. The method of claim 18 wherein said contacting the target tissue with the circumferentially extending tissue-cutting edge comprises:
positioning a blunt end of a stylet disposed within and extending distally from the distal end of the flexible elongate shaft adjacent to the target tissue; and
applying force to the device to cause the blunt end to move proximally into the flexible elongate shaft to allow the circumferentially extending tissue-cutting edge to contact the target tissue.

20. The method of claim 19, wherein the blunt end returns to a distal position in which the blunt end extends distally beyond the tissue-cutting edge once the tissue-cutting edge cuts through the target tissue.

21. The method of claim 20, further comprising axially advancing an outer sheath distally over the flexible elongate shaft and blunt end of the stylet such that the distal end of the flexible elongate shaft and the blunt end of the stylet are contained with the outer sheath.

22. The method of claim 18, further comprising, after the tissue-cutting edge has cut through the tissue, positioning an expandable member within a puncture hole cut in the tissue by the circumferentially extending tissue-cutting edge, and expanding the expandable member to increase a size of the cut hole.
